(19) United States
(12) Patent Application Publication
(10) Pub. No.: US 2010/0311661 A1
(43) Pub. Date: Dec. 9, 2010

(54) USE OF COMPOUNDS DERIVED FROM CYCLOHEXIMIDE FOR THE TREATMENT OR PREVENTION OF, IN PARTICULAR, ISCHAEMIAS AND CARDIOPATHIES

(75) Inventors: Gerhard Küllertz, Halle (DE); Gunter Fischer, Halle (DE); Steffi Rau, Eilenburg (DE); Britta Erika Husse, Gorlitz (DE)

Correspondence Address: CONNOLLY BOVE LODGE & HUTZ LLP, 1875 EYE STREET, N.W., SUITE 1100, WASHINGTON, DC, 20006, US

(73) Assignee: Max Planck Gesellschaft Zur Forderung Der Wissenschaften E.V., Munchen (DE)

(21) Appl. No.: 12/675,034

(22) PCT Filed: Aug. 24, 2007
(86) PCT No.: PCT/EP07/07459

§ 371 Date: Aug. 30, 2010

(51) Int. Cl. A61K 38/05; A61K 31/45; A61P 9/10; A61P 13/12; A61P 1/16; A61P 1/00; A61P 9/00; A61P 11/00; A61P 1/18; A61P 19/02; A61P 29/00; A01N 1/02

(52) U.S. Cl. 514/16.4; 514/328; 514/319; 514/21.91; 514/323; 435/1.1

(57) ABSTRACT

The present invention relates to the use of certain derivatives of cycloheximide or the pharmaceutically acceptable salts thereof for the manufacture of a medicament for the treatment or prevention of ischaemias, of cardiopathies, of endothelial disorders, of traumata, of necroses, of pulmonary disorders, of vascular disorders, of forms of shock, of blood flow impairments or of stroke; or for the manufacture of a medicament for the treatment or prevention of diseases caused by ischaemia; or for the manufacture of a composition for the preservation and/or storage of transplants, especially of organs.

The present invention relates to the use of a compound of general formula (I)

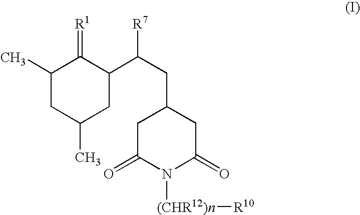

in which n is an integer of from 1 to 20;
in which R¹ is an O—, S—, NR²—, NOR²— or an N—NR²R³ residue,
   wherein R² is an H—, aryl or an alkyl residue which in the case of an aryl or of an alkyl residue can be interrupted by O or S or by an NH—, NR⁵—, aryl, heteroaryl, cycloalkyl or a heterocycloalkyl group or which in the case of an aryl or of an alkyl residue can carry a substituent R⁶, wherein R⁵ is an alkyl or an aryl residue and wherein R⁶ is an H—, alkyl, aryl, NH₂—, C(O)OR²—, OR⁵—, C(O)—, CN—, F— or Cl residue;
   wherein R³ independently of R² is an H—, aryl or an alkyl residue which in the case of an aryl or of an alkyl residue can be interrupted by O or S or by an NH—, NR⁵—, aryl, heteroaryl, cycloalkyl or a heterocycloalkyl group or which in the case of an aryl or of an alkyl residue can carry a substituent R⁶,
   wherein R⁵ is an alkyl or an aryl residue and wherein R⁶ is an H—, alkyl, aryl, NH₂—, C(O)OR²—, OR⁵—, C(O)—, CN—, F— or Cl residue;
   or wherein R² and R³ together form an alkylene residue with from 1 to 6 C atoms, which can be interrupted by O or S or by an NH—, NR⁵—, aryl, heteroaryl, cycloalkyl or a heterocycloalkyl group or which can carry an R⁶ residue as defined above;
in which R⁷ is an OH—, OR⁹—, OC(O)R⁹—, O(CHR¹²)ₙR^{10—}, OC(S)R⁹—, OC(O)NHR⁹— or an OC(S)NHR⁹ residue,
   wherein R⁹ is an alkyl residue which can be interrupted by O or S or by an NH—, NR⁵—, aryl, heteroaryl, cycloalkyl or a heterocycloalkyl group or which can carry an R⁶ residue as defined above;
   or wherein R⁹ is an aryl residue which can be interrupted by O or S or by an NH— or an NR⁵ group or which can carry an R⁶ residue as defined above;
in which R¹⁰ is an aryl, aryl-CN—, NHR²—, NR²R³—, C(O)OR²—, C(S)OR²—, C(O)NR²R³—, CN—, NR²C(O)NR²R³—, OC(O)NR²R³—, NR²C(S)NR²R³—, OC(S)NR²R³— or a C(O)NHR¹¹ residue,
   wherein R² and R³ are as defined above and R¹¹ is an amino acid residue or an oligopeptide residue;
      and in which R¹² is an H— or an alkyl residue;
      or of a physiologically acceptable salt thereof.

Cycloheximide (CHX) (4-[2-(3,5-dimethyl-2-oxocyclohexyl)-2-hydroxy-ethyl]-2,6-piperidindione; molecular weight: 281.34; melting point: 119-121° C.) was isolated from Streptomyces griseus in 1947 as an accompanying substance of streptomycin. Strongly diluted, it is effective against many yeasts, fungal skin diseases and parasitic fungi, such as, e.g., those causing brown spot in peaches, cherry leaf spot etc., less against bacteria (Lost, J. L., L. A. Kominek, G. S. Hyatt, and H. Y. Wang (1984) Cycloheximide: properties, biosynthesis, and fermentation; Van Dame E.J. (Ed.), Drugs and Pharmaceutical Sciences 22, 531-550). According to L.G. Jackson, L.G. and G.P. Studzinski (Autoradiographic studies of the effects of inhibitors of protein synthesis on RNA synthesis in HeLa cells. Exp. Cell Res. 52 (1968) 408-418), cycloheximide inhibits protein biosynthesis and has a toxic effect on eukaryotes (e.g.: B. Hardesty et al.: The effect of sodium fluoride, edeine, and cycloheximide on peptide synthesis with reticulocyte ribosomes. Basic Life Sci. 1, (1973) 377-392; T. Obrig et al: The mechanism by which cycloheximide and related glutarimide antibiotics inhibit peptide synthesis on reticulocyte ribosomes. J. Biol. Chem. 246 (1971) 174-181). Cycloheximide is used as fungicide in fruit-growing.

However, it was also shown that non-toxic doses of CHX can cause other additional effects: e.g. S. Mizuno et al. (Stress dose-dependent suppression of heat shock protein gene expression by inhibiting protein synthesis during heat shock treatment. Cell Struct. Funct. 22 (0.1997) 7-13) ascertained by means of kinetic analyses that the suppression of the induction of specific DNA sequences, called “heat shock” genes, by CHX can be distinguished from the effects produced by inhibiting protein synthesis in animal ova. This differentiated effect of CHX was also found in mRNA in tobacco cells. Thus Imanishi et al. (mRNA of tobacco cell, which is rapidly inducible by methyl jasmonate in the presence of cycloheximide, codes for a putative glycosyltransferase. Plant Cell Physiol. 39 (1998) 202-211) describe the selective. blocking or activation of specific mRNAs by CHX.

Numerous studies deal with the effects of CHX on neurons. Castagne and Clarke (Inhibition of glutathione synthesis can enhance cycloheximide-induced protection of developing neurons against axotomy. Brain Research. Developm. Brain Res. 102 (1997) 285-290) were able to show, by means of in vivo studies of retinal ganglion cells, that CHX inhibits cell death caused by axotomy. A. Kharlamov et al. (Cycloheximide reduces the size of lesion caused in rats by a photothrombotic model of brain injury. Neurolog. Res. 19 (1997) 92-96), who use a photothrombotic in vivo model in rats, also demonstrated that CHX reduces brain lesions which are usually triggered by radiation in this model. The protective property of CHX against cell death has also been demonstrated in the liver cells of rats (A. Sanchez et al.: Cycloheximide prevents apoptosis, reactive oxygen species production, and glutathione depletion induced by transforming growth factor beta in fetal rat hepatocytes in primary culture. Hepatology. 26 (1997) 935-943).

This therapeutically desirable effect of CHX, which ultimately leads secondarily to a reduction in cell damage caused by other primary factors, can be therapeutically used to only a limited extent, due to the high toxicity, described above, of CHX.

WO0026188 discloses compounds derived from cycloheximide. For these compounds, valuable pharmacological properties are described here which relate in particular to the healing of nerve damage caused by diseases or by surgical procedures and which are presumably based on the inhibition of the PPIase activity of enzymes of the FKBP12 type.

The object of the present invention is to find chemical compounds which have a favourable influence, promoting recovery and/or rehabilitation, on the myocardial state of myocardial infarction patients, and are therefore suitable for treating damage caused to the myocardium by myocardial infarction, as part of myocardial infarction and/or post-myocardial infarction treatment.

It was surprisingly found that the compounds mentioned at the outset are suitable for treating damage caused to the myocardium by myocardial infarction and that the compounds can also be used successfully in treating or preventing ischaemias, cardiopathies, endothelial diseases, traumata, necroses, pulmonary diseases, vascular diseases, forms of shock, blood supply disorders or strokes, for treating or preventing diseases caused by ischaemia or for preserving and/or storing transplants, in particular organs.

The present invention therefore relates to the use of a compound of general formula (I)

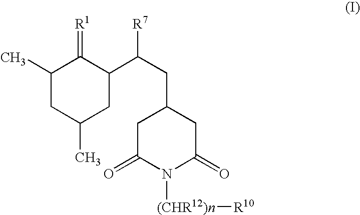

in which n is an integer of from 1 to 20;
in which R¹ is an O—, S—, NR²—, NOR²— or an N—NR²R³ residue,
   wherein R² is an H—, aryl or an alkyl residue which in the case of an aryl or of an alkyl residue can be interrupted by O or S or by an NH—, NR⁵—, aryl, heteroaryl, cycloalkyl or a heterocycloalkyl group or which in the case of an aryl or of an alkyl residue can carry a substituent R⁶, wherein R⁵ is an alkyl or an aryl residue and wherein R⁶ is an H—, alkyl, aryl, NH₂—, C(O)OR²—, OR⁵—, C(O)—, CN—, F— or Cl residue;
   wherein R³ independently of R² is an H—, aryl or an alkyl residue which in the case of an aryl or of an alkyl residue can be interrupted by O or S or by an NH—, NR⁵—, aryl, heteroaryl, cycloalkyl or a heterocycloalkyl group or which in the case of an aryl or of an alkyl residue can carry a substituent R⁶, wherein R⁵ is an alkyl or an aryl residue and wherein R⁶ is an H—, alkyl, aryl, NH₂—, C(O)OR²—, OR⁵—, C(O)—, CN—, F— or Cl residue;
   or wherein R² and R³ together form an alkylene residue with from 1 to 6 C atoms which can be interrupted by O or S or by an NH—, NR⁵—, aryl, heteroaryl, cycloalkyl or a heterocycloalkyl group or which can carry an R⁶ residue as defined above;
in which R⁷is an OH—, OR⁹—, OC(O)R⁹—, O(CHR¹²)ₙR¹⁰—, OC(S)R⁹—, OC(O)NHR⁹— or an OC(S)NHR⁹ residue,
   wherein R⁹ is an alkyl residue which can be interrupted by O or S or by an NH—, NR⁵—, aryl, heteroaryl, cycloalkyl or a heterocycloalkyl group or can carry an R⁶ residue as defined above;
   Or wherein R⁹ is an aryl residue which can be interrupted by O or S or by an NH— or an NR⁵ group or which can carry an R⁶ residue as defined above;
in which R¹⁰ is an aryl, aryl-CN—, NHR²—, NR²R³—, C(O)OR²—, C(S)OR²—, C(O)NR²R³—, CN—, NR²C(O)NR²R³—, OC(O)NR²R³—, NR²C(S)NR²R³—, OC(S)NR²R³— or a C(O)NHR¹¹ residue,
   wherein R² and R³ are as defined above and R¹¹ is an amino acid residue or an oligopeptide residue;

and in which R¹² is an H— or an alkyl residue;

or of a physiologically acceptable salt thereof

for manufacturing a medicament for treating or preventing ischaemias, cardiopathies, endothelial diseases, traumata, necroses, pulmonary diseases, vascular diseases, forms of shock, blood supply disorders or strokes;

or for manufacturing a medicament for treating or preventing diseases caused by ischaemia;

or for manufacturing a compound for preserving and/or storing transplants, in particular organs.

In general formula (I) and in the following an alkyl residue preferably means an alkyl residue with from 1 to 10 carbon atoms, such as for example a methyl, ethyl, propyl, isopropyl, cyclohexyl or an adamantyl residue which can carry one or more residues selected from the group consisting of aryl, heteroaryl, F, CN, NO₂, S, O and C(O). In general formula (I) and in the following cycloalkyl means in particular a C₄-C₇ cycloalkyl or a bi- or tricyclic system which can carry one or more residues selected from the group consisting of aryl, heteroaryl, F, CN, NO₂, S, O and C(O). Aryl means in particular phenyl or aryl substituted by alkyl, aryl, heteroaryl, F, CN, NO₂, C(O) and heteroaryl, in particular six-membered aromatics which contain nitrogen in the ring, or five-membered aromatics which contain nitrogen, oxygen or sulphur in the ring. Oligopeptide residues are in particular peptide residues with from 2 to 5 amino acid residues.

Suitable as physiologically acceptable salts are, for example, acid addition salts of inorganic acids, for example halogen hydracids, or of organic acids, for example lower aliphatic mono- or dicarboxylic acids such as acetic acid, fumaric acid or tartaric acid, or of aromatic carboxylic acids such as for example salicylic acid.

The compounds of general formula (I) can be manufactured in a manner known per se for example according to the methods described in the abovementioned international patent application or analogously to these methods.

It was surprisingly found that compounds of general formula (I) and their physiologically acceptable salts amongst other things have a favourable influence, promoting recovery and/or rehabilitation, on the myocardial state following a myocardial infarction, and are therefore suitable for treating damage caused to the myocardium by myocardial infarction in humans.

A subject of the present invention is also the anti-ischaemic effect of the compounds under discussion, in particular on the heart, in particular their use in prophylaxis, reinfarction prophylaxis, treatment of myocardial infarction and angina pectoris.

By a myocardial infarction is meant in general a necrosis of a circumscribed myocardial area due to continuous complete interruption of or critical reduction in the blood supply to this area. In addition to general therapeutic measures (analgesia and sedation, oxygen administration, bed rest and diet) in the case of acute myocardial infarction in particular a thrombolytic or fibrinolytic therapy is carried out with the aim of preserving as much (primary) ischaemic myocardium as possible from final cell death (i.e. definitive necrosis) by reperfusion of the ischaemic area and thus limiting the size of the infarct to as small an area as possible. Further (supporting) measures can also contribute to improving the myocardial condition, in particular around the infarction area, both in the acute phase of the myocardial infarction and in the post-myocardial infarction phase.

The compounds used according to the invention for treating damage caused to the myocardium by myocardial infarction are at the same time generally suitable for use as part of treating a myocardial infarction. They can therefore already be used when treating an acute myocardial infarction but in particular as part of a post-myocardial-infarction treatment both in patients who have already undergone successful fibrinolytic treatment and in patients without such a lysis. In the case of post-infarction patients with lysis, the treatment with the compounds used according to the invention in particular also has a prophylactic effect against the development of cardiac insufficiency caused by the myocardium (myocardial insufficiency). This also applies to patients who have already been treated with adrenoreceptor blockers.

For treatment according to the invention of damage caused to the myocardium by myocardial infarction, the compounds of general formula (I) or physiologically acceptable salts thereof can be administered orally, intravenously or also transdermally in conventional pharmaceutical preparations.

Thus the compounds and their salts can be contained, in a quantity promoting recovery and/or rehabilitation of the myocardial state, in solid or liquid pharmaceutical preparations together with conventional pharmaceutical adjuvants and/or supports. As examples of solid preparations which can be formulated for direct or delayed active ingredient release, preparations which can be used orally such as tablets, coated tablets, capsules, powders or granules may be named, or also suppositories and plasters (transdermal therapeutic systems). These solid preparations can contain pharmaceutically conventional inorganic and/or organic supports such as, for example, lactose, talc or starch in addition to pharmaceutically conventional adjuvants, for example lubricants or tablet disintegrants. In the case of plasters the active ingredient is accommodated in an active ingredient reservoir, in particular e.g. an active ingredient matrix (e.g. a polymer matrix).

Liquid preparations such as solutions, suspensions or emulsions of the active ingredients can contain the conventional diluents such as water, oils and/or suspending agents such as polyethylene glycols and the like. Further additional adjuvants can be added, such as e.g. preservatives, taste correctors and the like.

The active ingredients can be mixed in a manner known per se and formulated with the pharmaceutical adjuvants and/or supports. The active ingredients can for example be mixed with the adjuvants and/or supports in customary manner and granulated wet or dry in order to manufacture solid dosage forms. The granules or powder can be poured directly into capsules or be pressed in customary manner to produce tablet cores. If desired, these can be coated in known manner. Plasters, or transdermal therapeutic systems can be constructed in the customary manner e.g. from covering film, active ingredient reservoir (self-adhesive or with additional adhesive layer) and tear-off film both as matrix-controlled and as membrane-controlled (i.e. equipped with additional control membrane) systems.

Thanks to their discovered anti-ischaemic properties, the compounds of general formula (I) are also effective in the case of diseases such as may result from oxygen deficiency phenomena, such as for example in the case of blood supply disorders, wherein they can also preventatively inhibit or greatly reduce the pathophysiological processes when ischaemically induced damage occurs, in particular when ischaemically induced heart arrhythmia are triggered.

By blood supply disorders are meant both disturbances to the blood flow in arms and legs and also any diseases due to reduced blood supply in a tissue, organ or part of the body.

Blood supply disorders can result from narrowing or shifting of either the arterial inflow or the venous outflow. Causes of a blood supply disorder can be inflammations, vasoconstrictions (e.g. in the case of arteriosclerosis) or thromboses but can also include incorrect regulations of the vessel width as in the case of angina pectoris vasomotorica. If the blood supply disorder affects the heart, this can generally be referred to as myocardial infarction; if the impairment affects the brain, as a stroke; if the disorder affects the eyes, as retinopathy; if the disorder affects the lung, as pulmonary embolism; if the disorder affects the kidneys, this can be referred to as kidney failure; if the disorder affects the intestine, this can be referred to as intestinal malfunction. Irrespective of the internal organ affected by the blood supply disorder, such as, for example, heart, lung, liver, brain, spinal cord, intestine, or external organ such as, e.g., nose, ear, eyes, area of the body or part of the body, such as, e.g., the extremities or parts thereof, such as, for example, tissue (skin), damage caused by or to be expected due to ischaemia can be therapeutically influenced by medicinal products which contain compounds of formula (I). By therapeutic influence is meant both the healing effect on diseases and injuries and also the preventative effect on diseases and injuries to be expected in order to prevent the latter or to reduce their consequences.

By blood supply disorders are also meant within the meaning of the present invention states of organs and tissues which, although they have a regular blood flow, are also known, because of diseases due to oxygen supply problems, such as e.g. in the case of respiratory problems covered by the terms chronic obstructive pulmonary disease (COPD), acute respiratory distress syndrome (ARDS) or cystic fibrosis, to suffer ischaemic damage.

The extent of and degree of danger posed by a blood supply disorder depend mainly on the site and progress of the occlusion, the possible creation of an adequate parallel blood flow and on the general circulation situation.

Thanks to their protective effects vis-à-vis pathological hypoxic and ischaemic situations, the compounds of general formula (I) to be used can be used as medicinal products for treating all acute or chronic damage caused by ischaemia or diseases primarily or secondarily triggered by same.

Thanks to their protective effects vis-à-vis pathological hypoxic and ischaemic situations, the substances of general formula (I) can be used as medicinal products for treating coronary cardiopathy or ischaemic cardiopathy or for treating inflammatory diseases of the cardiovascular system or the myocardial muscle (myocarditis).

The compounds under discussion are also particularly suitable for use as medicinal products in surgical procedures, e.g. in the case of organ or tissue transplantations, wherein the substances can be used to protect the organs or tissue in the donor before and after removal, to protect the removed organs or tissue, for example in the case of treatment therewith or storage thereof in physiological bath liquids, as well as during transfer into the recipient organism. In addition to organ or tissue transplantation, it is often necessary during surgical procedures to temporarily reduce all or part of the blood flow in individual tissues or organs. Here the compounds can be used particularly well as medicinal products in order to have a preventative therapeutic influence on ischaemic damage to the affected tissue or organs by administering them beforehand, or subsequently administering them afterwards, in the case of reperfusion.

The compounds are also valuable medicinal products with a protective effect when carrying out angioplastic operations, for example on the heart, as well as on peripheral vessels, in the case of thromboembolic diseases such as thrombosis, myocardial infarction, arteriosclerosis, inflammations, stroke, angina pectoris, restenosis following angioplasty and intermittent claudication.

A further field of use of the compounds of general formula (I) are diseases which are described as multiple organ failure.

Multiple organ failure occurs for example when ischaemic damage to one organ leads to ischaemic damage to another organ. The liver e.g. receives most of the blood from the intestine. Thus ischaemia of the intestine subsequently often leads to ischaemia of the liver. The damaged liver can subsequently release numerous substances and enzymes such as, e.g., xanthine oxidase (XO). As the blood from the liver chiefly enters the lung, oxygen residues can be produced by the xanthine oxidase in the oxygen-rich lung, which can in turn lead to oxidative damage to the lung. A field of use of the present compounds can thus also be the treatment or prevention of oxidative damage to organs or tissues.

Thus the compounds under discussion can also be used as medicinal products in the case of male sterility, as oxidative stress can occur here in combination with high xanthine oxidase concentrations (M. Kurpisz et al.: Hum. Reprod. 11 (1996) 1223-6; D. Sanocka et al.: J. Androl. 17 (1996) 449-54).

Thanks to their protective properties vis-à-vis cellular ischaemia the named compounds are eminently suitable as medicinal products for treating ischaemias of the nervous system and in particular of the central nervous system, such as, for example, for treating strokes or cerebral oedema.

Thanks to their protective properties vis-à-vis cellular ischaemia the active ingredients are also particularly well suited as medicinal products to treat forms of shock, such as e.g. allergic, cardiogenic, hypovolaemic or bacterial shock.

Thanks to their protective properties vis-à-vis cellular ischaemia the active ingredients are also eminently suitable for use as medicinal products against muscle injuries resulting from sporting activity.

A further valuable property of the compounds which is based on the protective effect vis-à-vis cellular ischaemia is their effect as medicinal products against pain caused by cellular ischaemia. In particular the compounds are effective as medicinal products against pain covered by the term migraine.

Furthermore, compounds of general formula (I) also lead as medicinal products to a clear reduction in infarctions caused by metabolic anomalies, in particular to a significant reduction in the size of the induced infarct and its degree of severity.

Thanks to their protective property vis-à-vis cellular ischaemia the compounds under discussion are eminently suitable as medicinal products for treating ischaemias of the endothelial cells and thus endothelial damage. With this protection of the vessels against endothelial dysfunction syndrome, compounds of formula (I) are valuable medicinal products for preventing and treating coronary vessel spasms, atherogenesis, and atherosclerosis, left ventricular hypertrophy and dilated cardiomyopathy and thrombotic diseases.

Ischaemic damage can, however, also result from necessary surgical procedures or necessary therapy in the most varied diseases or accidents, or from these diseases or accidents themselves, such as, e.g. in the case of burns or frostbite or following pancreatitis, organ transplantations, cardiopathies, COPD or ARDS, cystic fibrosis, IBD (inflammatory bowel disease), cardiovascular collapse, metabolic arthritis (gout), RA (rheumatoid arthritis), OA (osteo-arthritis) or as a result of very varied liver diseases.

As the pharmaceutical effectiveness of the racemates or individual stereoisomers of the compounds of general formula (I) can differ, it can be desirable to use individual isolated stereoisomers. In these cases the end-product or else even the intermediate product in stereoisomerically pure compounds can be separated by chemical or physical measures known to a person skilled in the art or even used as such in the synthesis. In the case of racemic amines for example, diastereomers are formed from the mixture by reaction with an optically active separating agent. Suitable as separating agents are e.g. optically active acids, such as the R- and S-forms of tartaric acid, diacetyl tartaric acid, dibenzoyl tartaric acid, mandelic acid, malic acid, lactic acid, suitably N-protected amino acids (e.g. N-benzoyl proline or N-benzol sulphonyl proline) or the various optically active camphor sulphonic acids. Chromatographic enantiomer separation using an optically active separating agent (e.g. dinitrobenzoyl phenylglycine, cellulose triacetate or other hydrocarbon derivates or chirally derivatized methacrylate polymers fixed on silica gel) is also advantageous. Aqueous or alcoholic solvent mixtures, such as e.g. hexane/isopropanol/acetonitrile e.g. in the ratio 82:15:3 are also suitable as mobile phases for this.

A subject of the invention is also the use of compounds according to general formula (I) during reperfusion therapy. By reperfusion therapy is meant the restoration of the blood supply in the infarction area.

According to a preferred embodiment of the use according to the invention it is provided that n is 1, 2 or 3, R¹ is an O residue, R⁷ is an OH—, O(CHR¹²)ₙR¹⁰— or an OC(O)CH₃ residue and that R¹⁰ is a C(O)OCH₃—, C(O)OC₂H₅—, CN— or a C(O)NH₂ residue.

According to a further preferred embodiment of the use according to the invention it is provided that n is an integer of from 3 to 10, R¹ is an O residue, R⁷ is an OH residue and that R¹⁰ is a C(O)NHR¹¹ residue.

According to a preferred embodiment of the use according to the invention it is further provided that n is 1, 2 or 3, R¹ is an O residue, R⁷ is an OH— or an O(CHR¹²)ₙR¹⁰ residue and that R¹⁰ is a C(O)OCH₃—, C(O)OC₂H₅—, CN— or a C(O)NH₂ residue.

In another preferred embodiment of the use according to the invention it is also provided that n is 1, 2 or 3, R¹ is an NOH—, N—NHPh-, N—NHCH₃—, N-alkyl or an N-benzyl residue, R⁷ is an OH— or an O(CHR¹²)ₙ R¹⁰ residue and that R¹⁰ is a C(O)OCH₃—, C(O)OC₂H₅—, CN— or a C(O)NH₂ residue.

According to a further preferred embodiment of the use according to the invention it is provided that n is 1, 2 or 3, R¹ is an O residue, R⁷ is an OH—, O(CHR¹²)ₙR¹⁰—, OC(O)NH-alkyl, OC(O)NH-cycloalkyl or an OC(O)NH-aryl residue and that R¹⁰ is a C(O)OCH₃—, C(O)OC₂H₅—, CN— or a C(O)NH₂ residue.

Particularly preferred within the framework of the present invention is a compound of general formula (I) in which n is equal to 1, R¹ is O, R⁷ is OH, R¹² is H and R¹⁰ is C(O)N(CH₃)₂. The compound is in particular N′,N′-dimethylcarboxyamidomethyl)-cycloheximide or a physiologically acceptable salt thereof. This compound is particularly suitable for treating damage caused to the myocardium by myocardial infarction as part of myocardial infarction and/or post-myocardial infarction treatment and accordingly preferred according to the invention.

According to another preferred embodiment of the use according to the invention it is provided that the compound is a compound selected from the following group of compounds:

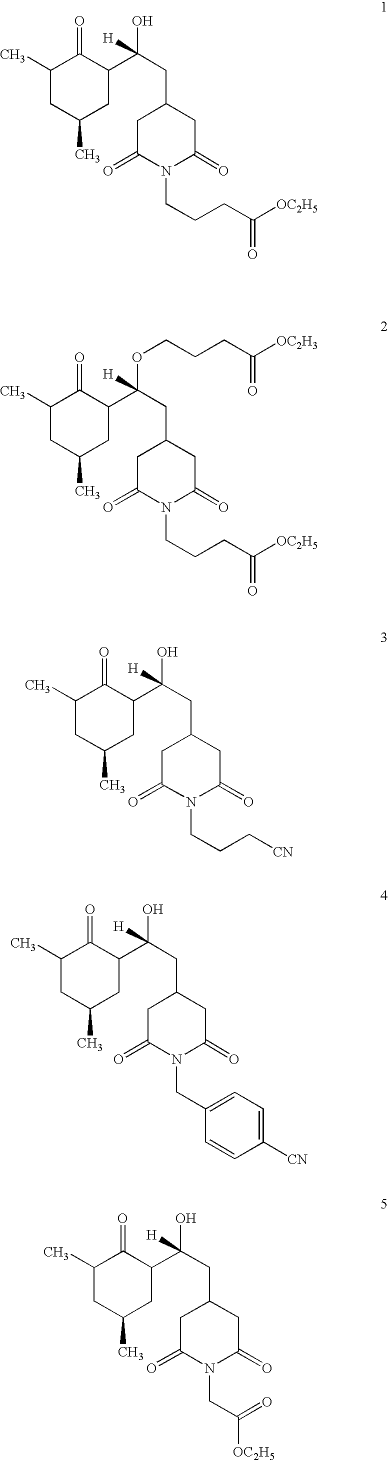

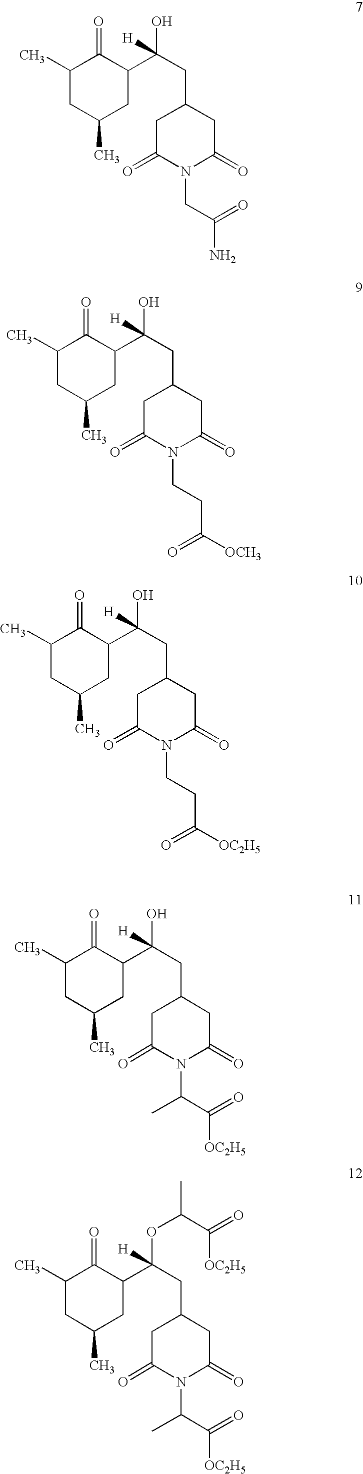

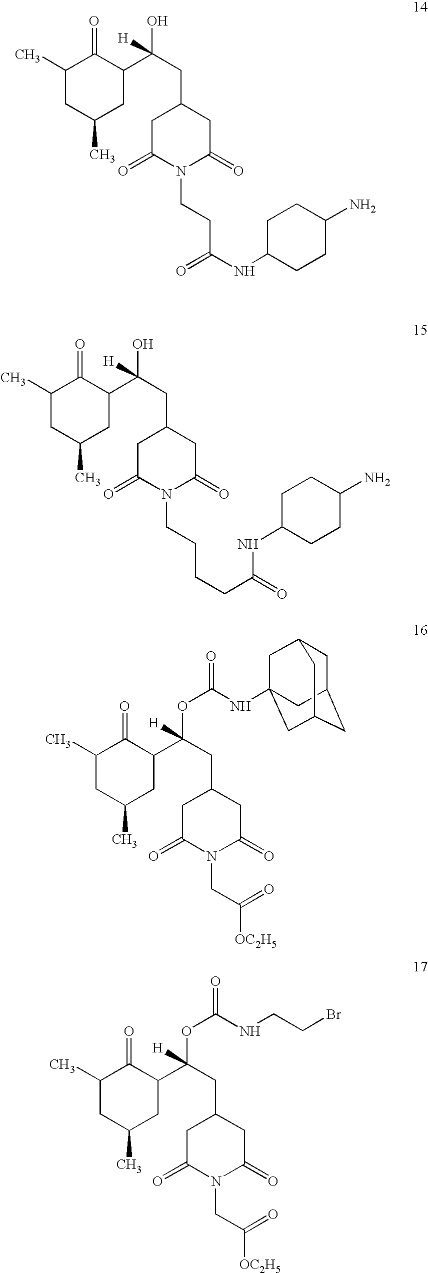

According to a preferred embodiment of the use according to the invention it is also provided that the compound is the compound of Formula 1.

According to a preferred embodiment of the use according to the invention it is further provided that the compound is the compound of Formula 2.

According to a preferred embodiment of the use according to the invention it is furthermore provided that the compound is the compound of Formula 3.

According to a further preferred embodiment of the use according to the invention it is provided that the compound is the compound of Formula 4.

According to a further preferred embodiment of the use according to the invention it is provided that the compound is the compound of Formula 5.

According to an additional preferred embodiment of the use according to the invention it is provided that the compound is the compound of Formula 7.

Furthermore according to a preferred embodiment of the use according to the invention it is provided that the compound is the compound of Formula 9.

According to a further preferred embodiment of the use according to the invention it is provided that the compound is the compound of Formula 10.

According to a preferred embodiment of the use according to the invention it is provided that the compound is the compound of Formula 11.

According to a preferred embodiment of the use according to the invention it is provided that the compound is the compound of Formula 12.

According to a further preferred embodiment of the use according to the invention it is provided that the compound is the compound of Formula 14.

According to another preferred embodiment of the use according to the invention it is provided that the compound is the compound of Formula 15.

According to an alternative preferred embodiment of the use according to the invention it is also provided that the compound is the compound of Formula 16.

According to a preferred embodiment of the use according to the invention it is further provided that the compound is the compound of Formula 17.

According to another preferred embodiment of the use according to the invention it is provided that the compound is a compound of general formula (II)

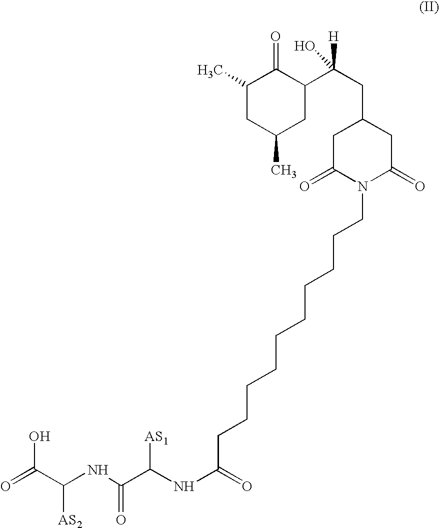

in which the AS₁ residue is the side chain of the amino acid alanine, valine, tryptophan, isoleucine or methionine or H and in which the AS₂ residue is the side chain of the amino acid alanine or valine. It can be particularly preferred if the stereogenic centres in the peptide fragment of the compound are L-configured.

According to a preferred embodiment of the use according to the invention it is further provided that the AS₁ residue is the side chain of the amino acid alanine and that the AS₂ residue is the side chain of the amino acid alanine.

According to a preferred embodiment of the use according to the invention it is also provided that the AS₁ residue is the side chain of the amino acid valine and that the AS₂ residue is the side chain of the amino acid alanine.

According to a preferred embodiment of the use according to the invention it is also provided that the AS₁ residue is the side chain of the amino acid tryptophan and that the AS₂ residue is the side chain of the amino acid alanine.

According to a preferred embodiment of the use according to the invention it is further provided that the AS₁ residue is the side chain of the amino acid isoleucine and that the AS₂ residue is the side chain of the amino acid alanine.

According to a further preferred embodiment of the use according to the invention it is provided that the AS₁ residue is the side chain of the amino acid methionine and that the AS₂ residue is the side chain of the amino acid alanine.

According to a preferred embodiment of the use according to the invention it is further provided that the AS₁ residue is an H residue and that the AS₂ residue is the side chain of the amino acid alanine.

According to a preferred embodiment of the use according to the invention it is also provided that the AS₁ residue is the side chain of the amino acid alanine and that the AS₂ residue is the side chain of the amino acid valine.

According to a further preferred embodiment of the use according to the invention it is provided that the AS₁ residue is the side chain of the amino acid valine and that the AS₂ residue is the side chain of the amino acid valine.

According to a preferred embodiment of the use according to the invention it is also provided that the AS₁ residue is the side chain of the amino acid tryptophan and that the AS₂ residue is the side chain of the amino acid valine.

According to a preferred embodiment of the use according to the invention it is further provided that the AS₁ residue is the side chain of the amino acid isoleucine and that the AS₂ residue is the side chain of the amino acid valine.

According to a preferred embodiment of the use according to the invention it is further provided that the AS₁ residue is the side chain of the amino acid methionine and that the AS₂ residue is the side chain of the amino acid valine.

According to a further preferred embodiment of the use according to the invention it is provided that the AS₁ residue is an H residue and that the AS₂ residue is the side chain of the amino acid valine.

According to a preferred embodiment of the use according to the invention it is further provided that the compound is a compound selected from the following group of compounds:

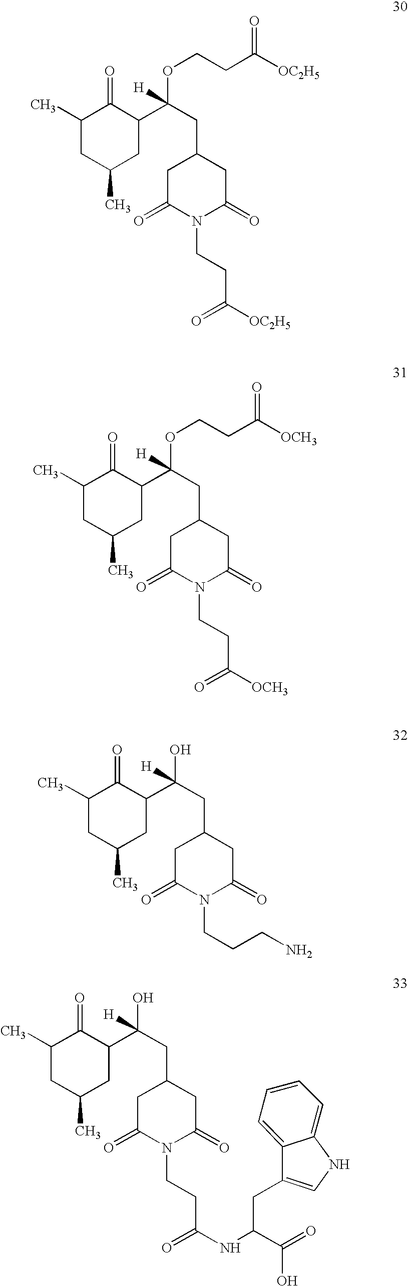

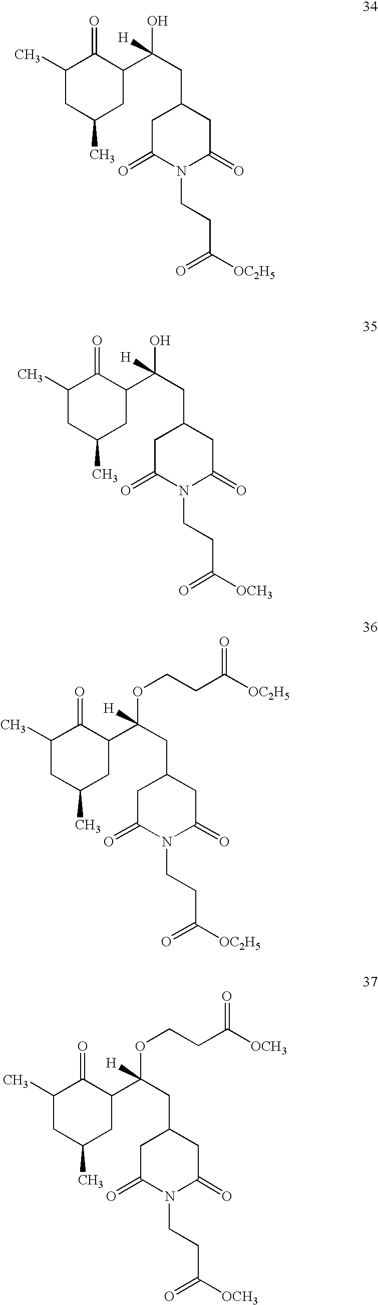

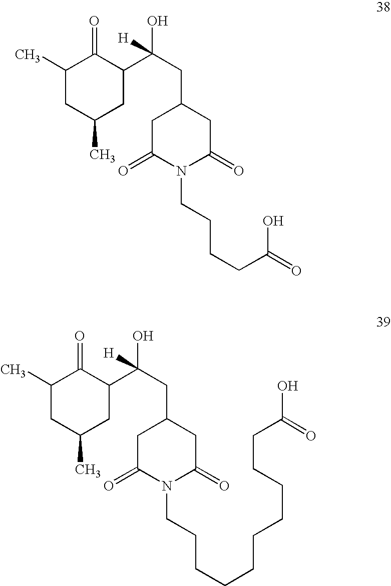

According to a preferred embodiment of the use according to the invention it is provided that the compound is the compound of Formula 30.

According to another preferred embodiment of the use according to the invention it is provided that the compound is the compound of Formula 31.

According to another preferred embodiment of the use according to the invention it is provided that the compound is the compound of Formula 32.

According to a preferred embodiment of the use according to the invention it is further provided that the compound is the compound of Formula 33.

According to a further preferred embodiment of the use according to the invention it is provided that the compound is the compound of Formula 34.

According to a further preferred embodiment of the use according to the invention it is provided that the compound is the compound of Formula 35.

According to a further preferred embodiment of the use according to the invention it is provided that the compound is the compound of Formula 36.

According to a preferred embodiment of the use according to the invention it is further provided that the compound is the compound of Formula 37.

According to a preferred embodiment of the use according to the invention it is further provided that the compound is the compound of Formula 38.

According to a further preferred embodiment of the use according to the invention it is provided that the compound is the compound of Formula 39.

According to a preferred embodiment of the use according to the invention it is also provided that the ischaemia is cardiac ischaemia, hepatic ischaemia, renal ischaemia, intestinal ischaemia or cerebral ischaemia.

According to a preferred embodiment of the use according to the invention it is further provided that the cardiopathy is damage caused to the myocardium by myocardial infarction, coronary insufficiency, myocardial infarction, angina pectoris, a coronary cardiopathy, an inflammatory disease of the cardiovascular system or of the myocardial muscle, in particular myocarditis, left-ventricular hypertrophy or dilated cardiomyopathy, in particular damage caused to the myocardium by myocardial infarction.

According to a further preferred embodiment of the use according to the invention it is provided that the endothelial disease is damage to the endothelium, in particular damage to the endothelium caused by traumata, or an endothelial dysfunction.

According to a preferred embodiment of the use according to the invention it is also provided that the traumata are traumata caused by surgical procedures, in particular traumata caused by transplantations, in particular traumata caused by angioplasty operations.

According to a further preferred embodiment of the use according to the invention it is provided that the necrosis is a necrosis of the brain, heart, liver, kidney or intestinal cells/tissue.

According to a preferred embodiment of the use according to the invention it is further provided that the lung disease is a chronic obstructive pulmonary disease, acute respiratory distress syndrome or mucoviscidosis.

According to a further preferred embodiment of the use according to the invention it is provided that the vascular disease is angiospasm, in particular angiospasm in the case of Raynaud's disease, Prinzmetal's angina or in the case of ergotism, or coronary vasospasm, atherogenesis, atherosclerosis or a thrombotic disease.

According to a preferred embodiment of the use according to the invention it is furthermore provided that the form of the shock is an allergic, hypovolaemic or bacterial shock.

According to a preferred embodiment of the use according to the invention it is also provided that the diseases caused by ischaemia are ischaemic cell or tissue damage, preferably necroses, in particular necroses of the heart, brain, liver, kidney or intestinal cells or tissues, or preferably of traumata, burns, frostbite, pancreatitis, transplantations, cardiopathies, pulmonary diseases such as COPD, ARDS, cystic fibrosis or pulmonary infarction, chronic inflammatory intestinal diseases such as Crohn's disease, cardiovascular collapse, metabolic arthritis, rheumatoid arthritis, osteoarthritis or ischaemic cell or tissue damage caused by liver diseases.

According to a further preferred embodiment of the use according to the invention it is provided that the diseases caused by ischaemia are myocardial infarction or stroke or ischaemic diseases or heart conditions.

According to a preferred embodiment of the use according to the invention it is also provided that the diseases caused by ischaemia are ischaemic conditions of the peripheral and central nervous system or ischaemic conditions following a stroke, in particular ischaemic conditions of the brain.

According to a preferred embodiment of the use according to the invention it is further provided that the diseases caused by ischaemia are ischaemic conditions which trigger pain, in particular migraine.

According to a preferred embodiment of the use according to the invention it is also provided that the diseases caused by ischaemia are ischaemic conditions of peripheral organs or limbs.

Compounds of formula (I) can be applied orally, parenterally, intravenously, rectally or by inhalation, wherein the preferred application depends on the symptoms of the disease in question. The compounds can be used alone or together with galenic adjuvants, in veterinary as well as in human medicine.

A person skilled in the art is familiar with which adjuvants are suitable for the desired medicinal product formulation. In addition to solvents, gelling agents, suppository bases, tablet adjuvants and other active ingredient supports, antioxidants, dispersants, emulsifiers, anti-foaming agents; taste correctors, preservatives, solubilizers or colouring agents can be used for example.

For an oral application form the active compounds are mixed with the additives suitable for the purpose, such as supports, stabilizers or inert diluents and made up into the suitable administration forms such as tablets, coated tablets, hard-shell capsules, aqueous, alcoholic or oily solutions by the usual methods. There can be used as inert carriers e.g. gum arabic, magnesia, magnesium carbonate, potassium phosphate, lactose, glucose or starch, in particular maize starch. The preparation can be as both dry and moist granules. Plant or animal oils, such as sunflower oil or cod liver oil, may be considered for example as oily supports or as solvents.

For subcutaneous or intravenous application the medicinal products which contain a compound of Formula (I) or a salt thereof are, if desired, dissolved, suspended or emulsified with the conventional substances for this, such as solubilizers, emulsifiers or further adjuvants. The following are considered as solvent for example: water, physiological saline solution or alcohols, for example ethanol, propanol or glycerine, in addition also sugar solutions such as glucose or mannitol solutions, or also a mixture of the different named solvents.

The following are suitable for example as pharmaceutical formulation for administration in the form of aerosols or sprays: solutions, suspensions or emulsions of the active ingredients, such as in particular ethanol- or water-containing mixtures.

The formulation can, as required, also contain further other pharmaceutical adjuvants such as surfactants, emulsifiers and stabilizers as well as a propellant gas. Such a preparation usually contains the active ingredient in a concentration of approximately from 0.1 to 10, in particular of approximately from 0.3 to 3 wt.-%.

The dosage of the medicinal product to be administered which contains a compound of Formula (I) and the frequency of administration depend on the potency and duration of effect of the compounds used, and also on the nature and strength of the disease to be treated as well as on the sex, age, weight and individual responsiveness of the mammal to be treated.

On average the daily dose of a compound of Formula (I) or of a salt thereof for a patient weighing approximately 75 kg is at least 0.001 mg/kg, preferably at least 0.01 mg/kg, in particular at least 0.1 mg/kg up to at most 10 mg/kg, preferably at most 1 mg/kg body weight. During acute episodes of the disease, for example immediately after suffering a myocardial infarction, even higher and, especially, more frequent doses may also be necessary, e.g. up to 4 single doses daily. In particular in the case of i.v. application, for example in the case of an infarction patient in intensive care, up to 200 mg daily may be necessary.

In a further application, administration forms which contain a compound of Formula (I) can contain the active ingredient in the form of nanoparticles. Nanoparticles are usually 30-1000 nm in diameter, spherical or non-spherical in shape and frequently polymer-based. The compounds under discussion can be embedded in the nanoparticles or uniformly or non-uniformly dispersed in the polymer matrix, or else adsorbed at the surface of the nanoparticles or also contain these in combinations of these forms.

Nanoparticles are particularly suitable as, due to their small size, they can be taken up by phagocytic cells such as, for example, monocytes or macrophages. In a particularly preferred application the polymer used is biocompatible and biodegradable poly(DL-lactide-co-glycolide) polymer (PLGA). However, other polymers or mixtures of polymers or nanoparticles manufactured from these can also be used.

The following embodiment examples serve to explain the invention in conjunction with the drawing. There are shown in:

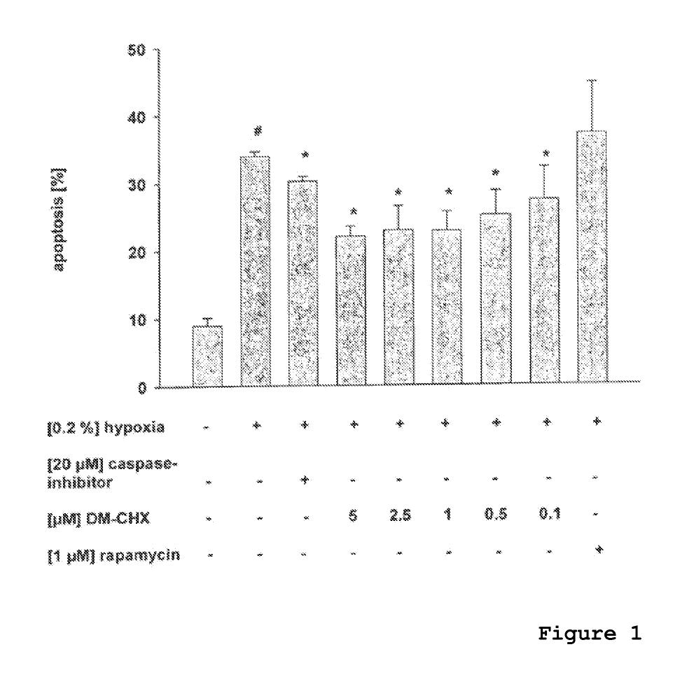

FIG. 1: Effect of DM-CHX on ischaemia of adult heart cells triggered by hypoxia, shown as a histogram;

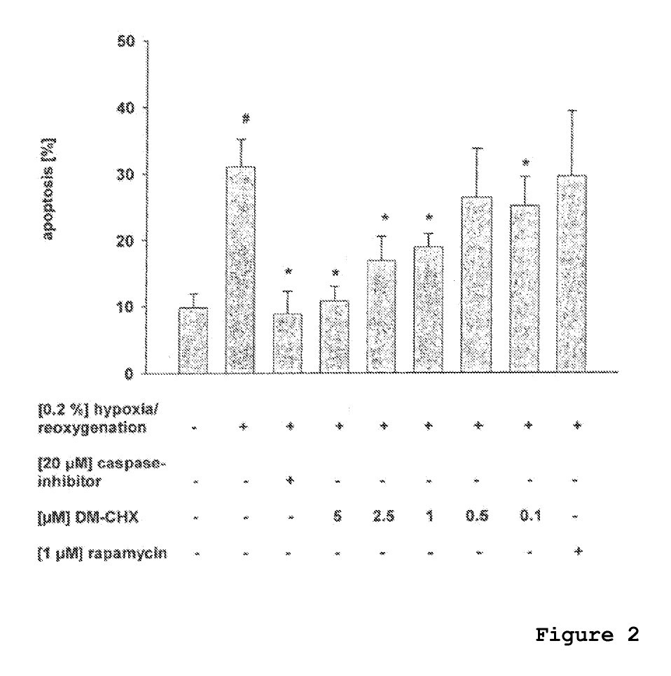

FIG. 2: Effect of the compound DM-CHX on adult cardiomyocytes influenced by hypoxia and subsequent reoxygenation, shown as a histogram;

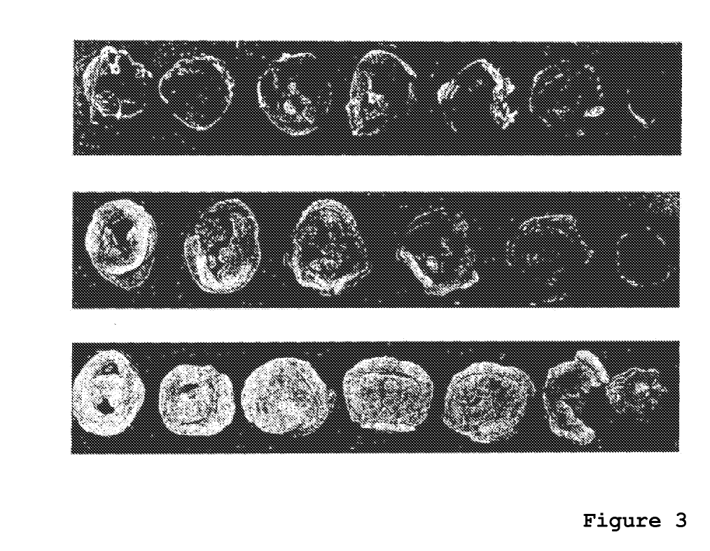

FIG. 3: Histological sections of hearts supplied with oxygen-saturated buffer (top row); histological sections of hearts supplied with reperfusion buffer with 50 μM DM-CHX (middle row); histological sections of hearts supplied with reperfusion buffer without DM-CHX (bottom row);

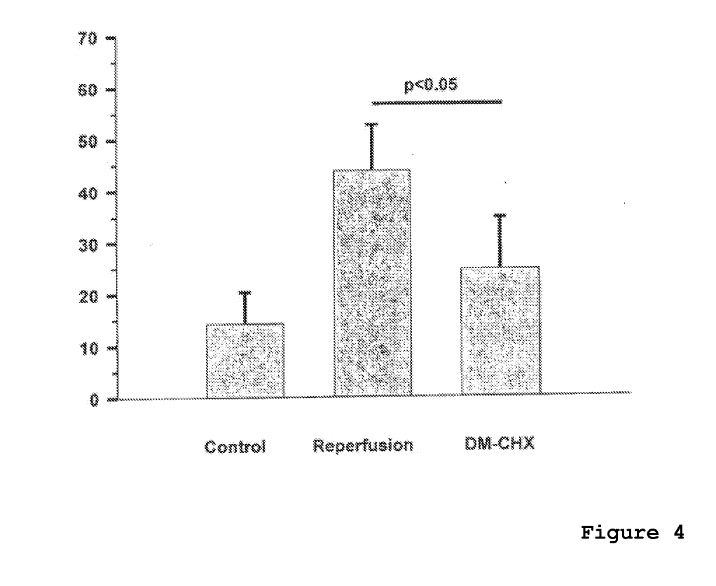

FIG. 4: Qualitative result of the size of the infarct, shown as a histogram.

In the embodiment examples 1-3 the cycloheximide derivative, here called DM-CHX, corresponding to general formula (I) with R¹═O, R⁷═OH—, R¹²═H, n=l and R¹⁰ ═N(CHA₂-C(O)— was used. In Examples 1 and 2 the caspase inhibitor Z-VAD-FMK, frequently used in research, was used. As intracellular caspase activation is a key event in cell apoptosis, this inhibitor is used by numerous authors (e.g.: Piguet P. F. et al.: Laboratory Investigation. 79 (1999) 495-500; Lavoie J. N. et al.: Journal of Cell Biology. 140 (1998) 637-645) to suppress apoptosis.

### EXAMPLE 1

Example 1 relates to the effect of DM-CHX on ischaemia of adult heart cells triggered by hypoxia.

Adult myocytes were accordingly isolated from 9-week-old male animals (Sprague-Dawley) (Husse B. Sopart A. Isenberg G. American Journal of Physiology—Heart & Circulatory Physiology. 285(4):H1521-H1527, 2003) and subjected to a maintenance culture in medium M199 (Sigma). The cells were placed in an incubator for 18 hours, setting the nitrogen/oxygen concentration of the incubator air to an oxygen concentration of 0.2%. Immediately before hypoxic conditions were introduced, the active ingredients (examples 1-2) were added. The percentage influence on the cells, expressed as apoptosis, was determined by means of Guava ViaCount assay. The left-hand bar in FIG. 1. shows the natural apoptosis of the cells ascertained with the method under the chosen conditions. It can be clearly seen that the PPIase inhibitor rapamycin does not influence the percentage apoptosis. On the other hand, concentrations of DM-CHX greater than 0.1 μM display much stronger effects in the reduction of the percentage apoptosis than the caspase inhibitor Z-VAD-FMK used for comparison in a concentration of 20 μM. Significant differences (p<0.05) between the individual experiments were calculated by means of T-test compared with the hypoxic control (*) or normoxic control (#). The error bars correspond to the standard deviation calculated from the reproduction of 5 independent experiments.

### EXAMPLE 2

Example 2 relates to the effect of the active ingredient DM-CHX on the adult cardiomyocytes influenced by hypoxia and subsequent reoxygenation.

Adult myocytes were isolated as in Example 1.The cells were subjected to hypoxic conditions of 0.2% oxygen for 18 hours as described in example 1, and then normoxic conditions for 24 hours. Apoptosis was determined as described in example 1. The left-hand bar in FIG. 2 shows the natural apoptosis of the cells ascertained with the method under the chosen conditions. It can be clearly seen that the PPIase inhibitor rapamycin has hardly any influence on the apoptosis obtained after reperfusion. On the other hand, concentrations of DM-CHX greater than 1 μM display a statistically significant influence corresponding at a concentration of 5 μM to the caspase inhibitor Z-VAD-FMK used for comparison in a concentration of 20 μM. Under the chosen test conditions 1 μM DM-CHX leads to an apoptosis rate which corresponds to the untreated cells (left-hand bar). Significant differences (p<0.05) between the individual experiments were calculated by means of T-test compared with hypoxic control (*) or normoxic control (#). The error bars correspond to the standard deviation calculated from the reproduction of 5 independent experiments.

### EXAMPLE 3

Example 3 relates to the effect of the size of the infarct on an ischaemia/reperfusion model.

Hearts of 9-week-old male rats (Sprague-Dawley) were examined using a Langendorff perfusion apparatus. This apparatus makes possible the retrograde perfusion of the heart (perfusion flow 1.8 ml/min). In a first period the heart was washed free of blood with a Ca²⁺-free solution (Tyrode). The perfusion protocol starts with a 15-minute oxygen-saturated perfusion period, the perfusion buffer containing: 5 μM HEPES, 150 mM NaCl, 4.2 mM KCl, 1.2 mM MgSO₄x7H₂O, 1.2 mM KH₂PO₄, 10 mM glucose and 1.8 mM CaCl₂x2H₂O (pH 7.4). Global ischaemia is then created by stopping the perfusion pump for 20 min. In the third period the reperfusion is resumed with the oxygen-saturated buffer for two hours.

The three groups in FIG. 3 differ as follows:

Group 1 (top row): Here the perfusion pump has not been stopped. These hearts were supplied with the oxygen-saturated buffer for 2 hours and 20 min.

Group 2 (middle row): Here global ischaemia has been created and 50 μM DM-CHX added to the reperfusion buffer.

Group 3 (bottom row): Here global ischaemia has been created and no DM-CHX added to the reperfusion buffer.

After reperfusion the hearts were frozen and then cut into 2-3 mm-wide strips. To detect the size of the infarct the pieces were incubated for 22 min at 37° C. with 2,3,5-triphenyltetrazolium chloride solution (TTC, 1%) and then fixed with 4% formalin. FIG. 4 shows the qualitative result for the size of the infarct. The uncoloured areas of the hearts correspond to the damaged heart tissue areas. Visually, a serious difference in colouring is to be seen between the hearts treated with DM-CHX and the untreated hearts, both subjected to global ischaemia for 20 min.

Quantification was carried out with the Biophotonic Image Program. The result is shown in FIG. 4. With 4 different hearts for each test arrangement, it was possible, by means of T-Test, to calculate a significant difference between the reperfused hearts provided with added DM-CHX and the reperfused hearts without this addition.

1. A method for treating or preventing ischaemias, or for treating or preventing diseases caused by ischaemia, or for preserving and/or storing transplants; the method comprising administering a compound of general formula (I) 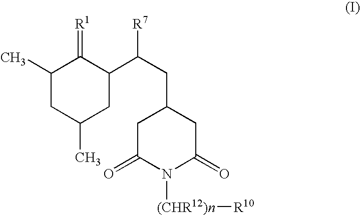
in which n is an integer of from 1 to 20;
in which R¹ is an O—, S—, NR²—, NOR²— or an N—NR²R³ residue,
wherein R² is an H—, aryl or an alkyl residue which in the case of an aryl or of an alkyl residue can be interrupted by O or S or by an NH—, NR⁵—, aryl, heteroaryl, cycloalkyl or a heterocycloalkyl group or which in the case of an aryl or of an alkyl residue can carry a substituent R⁶, wherein R⁵ is an alkyl or an aryl residue and wherein R⁶ is an H—, alkyl, aryl, NH₂—, C(O)OR²—, OR⁵—, C(O)—, CN—, F— or Cl residue;
wherein R³ independently of R² is an H—, aryl or an alkyl residue which in the case of an aryl or of an alkyl residue can be interrupted by O or S or by an NH—, NR⁵—, aryl, heteroaryl, cycloalkyl or a heterocycloalkyl group or which in the case of an aryl or of an alkyl residue can carry a substituent R⁶, wherein R⁵ is an alkyl or an aryl residue and wherein R⁶ is an H—, alkyl, aryl, NH₂—, C(O)OR²—, OR⁵—, C(O)—, CN—, F— or Cl residue;
or wherein R² and R³ together form an alkylene residue with from 1 to 6 C atoms which can be interrupted by O or S or by an NH—, NR⁵—, aryl, heteroaryl, cycloalkyl or a heterocycloalkyl group or which can carry an R⁶ residue as defined above;
in which R⁷ is an OH—, OR⁹—, OC(O)R⁹—, O(CHR¹²)ₙR¹⁰-, OC(S)R⁹—, OC(O)NHR⁹— or an OC(S)NHR⁹ residue,
wherein R⁹ is an alkyl residue which can be interrupted by O or S or by an NH—, NR⁵—, aryl, heteroaryl, cycloalkyl or a heterocycloalkyl group or which can carry an R⁶ residue as defined above;
or wherein R⁹ is an aryl residue which can be interrupted by O or S or by an NH— or an NR⁵ group or which can carry an R⁶ residue as defined above;
in which R¹⁰ is an aryl, aryl-CN—, NHR²—, NR²R³—, C(O)OR²—, C(S)OR²—, C(O)NR²R³—, CN—, NR²C(O)NR²R³—, OC(O)NR²R³—, NR²C(S)NR²R³—, OC(S)NR²R³— or a C(O)NHR¹¹ residue,
wherein R² and R³ are as defined above and R¹¹ is an amino acid residue or an oligopeptide residue;
and in which R¹² is an H— or an alkyl residue;
or of a physiologically acceptable salt thereof.
wherein the ischaemia is cardiac ischaemia, hepatic ischaemia, renal ischaemia or intestinal ischaemia, cardiopathies, endothelial diseases, traumata, necroses, wherein the necrosis is a necrosis of the heart, liver, kidney or intestinal cells/tissue, pulmonary diseases, vascular diseases, forms of shock, blood supply disorders or strokes; and
wherein the diseases caused by ischaemia are ischaemic cell or tissue damage, traumata, burns, frostbite, pancreatitis, transplantations, cardiopathies, pulmonary diseases such as COPD, ARDS, cystic fibrosis or pulmonary infarction, chronic inflammatory intestinal disease, cardiovascular collapse, metabolic arthritis, rheumatoid arthritis, osteoarthritis or ischaemic cell or tissue damage caused by liver diseases.

2. The method according to claim 1, wherein n is 1, 2 or 3, R¹ is an O residue, R⁷ is an OH—, O(CHR¹²)ₙR¹⁰— or an OC(O)CH₃ residue and R¹⁰ is a C(O)OCH₃—, C(O)OC₂H₅—, CN— or a C(O)NH₂ residue
or
n is an integer of from 3 to 10, R¹ is an O residue, R⁷ is an OH residue and R¹⁰ is a C(O)NHR¹¹ residue
or
n is 1, 2 or 3, R¹ is an O residue, R⁷ is an OH— or an O(CHR¹²)ₙR¹⁰ residue and R¹⁰ is a C(O)OCH₃—, C(O)OC₂H₅—, CN— or a C(O)NH₂ residue
or
n is 1, 2 or 3, R¹ is an NOH—, N—NHPh-, N—NHCH₃—, N-alkyl or an N-benzyl residue, R⁷ is an OH— or an O(CHR¹²)ₙ R¹⁰ residue and R¹⁰ is a C(O)OCH₃—, C(O)OC₂H₅—, CN— or a C(O)NH₂ residue
or
n is 1, 2 or 3, R¹ is an O residue, R⁷ is an OH—, O(CHR¹²)ₙR¹⁰—, OC(O)NH-alkyl, OC(O)NH-cycloalkyl or an OC(O)NH-aryl residue and R¹⁰ is a C(O)OCH₃—, C(O)OC₂H₅—, CN— or a C(O)NH₂ residue
or
n is equal to 1, R¹ is O, R⁷ is OH, R¹² is H and R¹⁰is C(O)N(CH₃)₂, the compound is preferably N′,N′-dimethylcarboxyamidomethyl)-cycloheximide.

3. The method according to claim 1, wherein the compound is a compound selected from the following group of compounds:

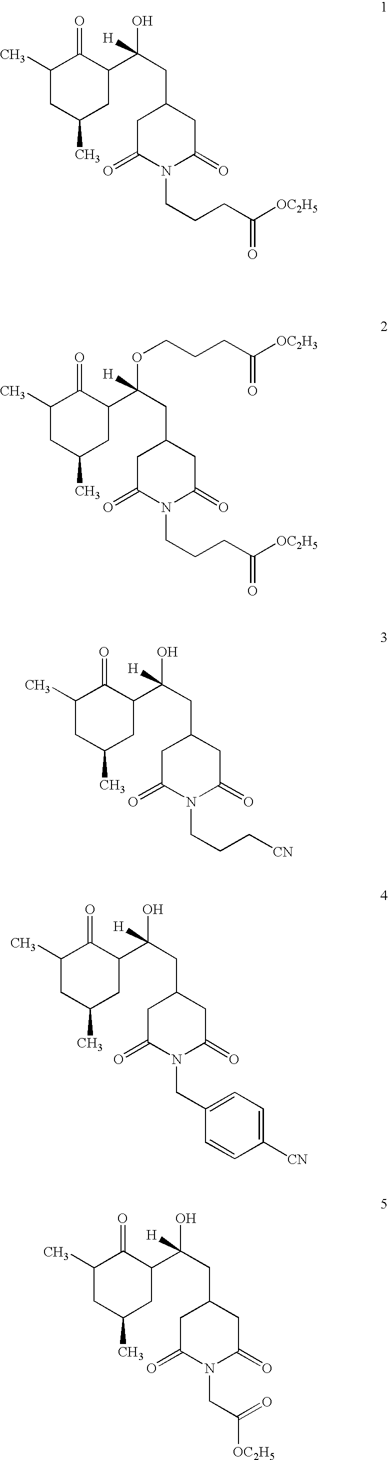

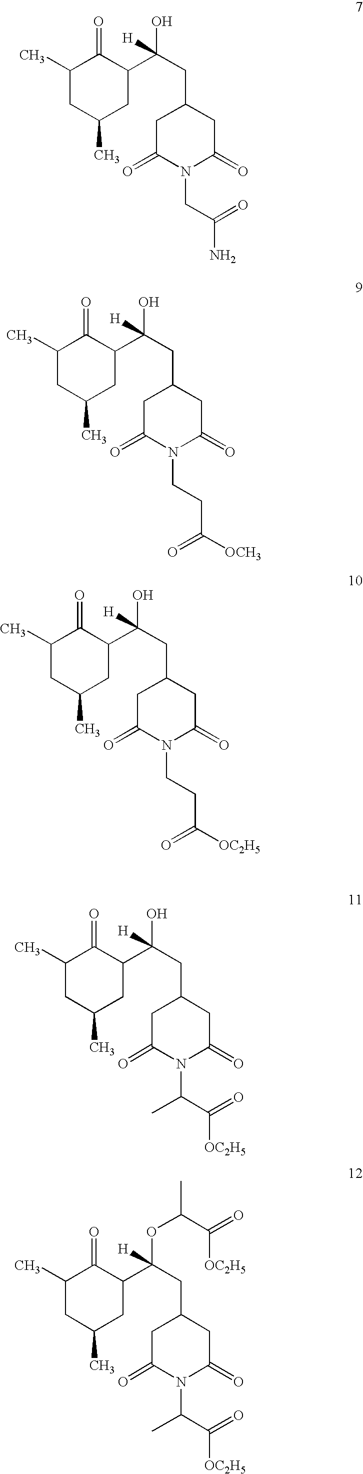

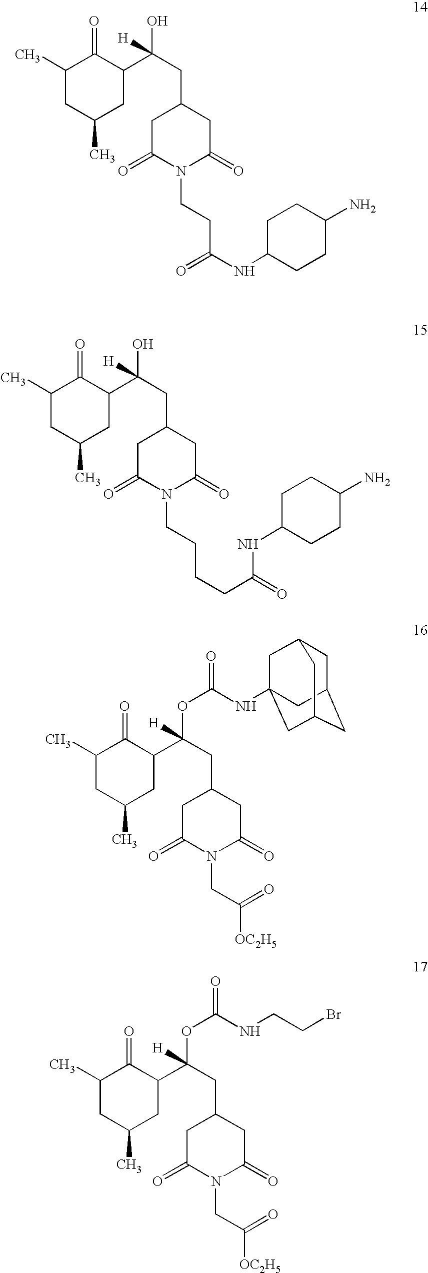

4. The method according to claim 1, wherein the compound is a compound of general formula (II) 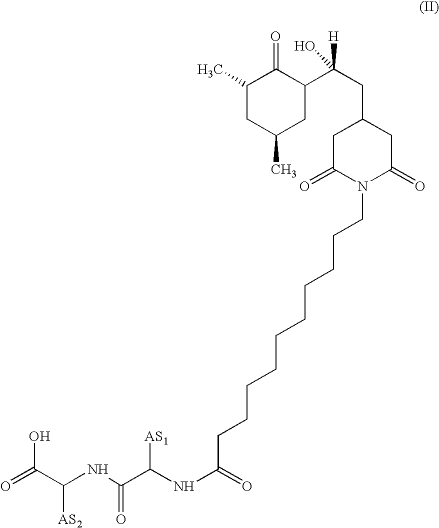
in which the AS₁ residue is the side chain of the amino acid alanine, valine, tryptophan, isoleucine or methionine or H and in which the AS₂ residue is the side chain of the amino acid alanine or valine.

5. The method according to claim 4, wherein the AS₁ residue is the side chain of the amino acid alanine and the AS₂ residue is the side chain of the amino acid alanine.

6. The method according to claim 4, wherein the AS₁ residue is the side chain of the amino acid valine and the AS₂ residue is the side chain of the amino acid alanine.

7. The method according to claim 4, wherein the AS₁ residue is the side chain of the amino acid tryptophan and the AS₂ residue is the side chain of the amino acid alanine.

8. The method according to claim 4, wherein the AS₁ residue is the side chain of the amino acid isoleucine and the AS₂ residue is the side chain of the amino acid alanine.

9. The method according to claim 4, wherein the AS₁ residue is the side chain of the amino acid methionine and the AS₂ residue is the side chain of the amino acid alanine.

10. The method according to claim 4, wherein the AS₁ residue is an H residue and the AS₂ residue is the side chain of the amino acid alanine.

11. The method according to claim 4, wherein the AS₁ residue is the side chain of the amino acid alanine and the AS₂ residue is the side chain of the amino acid valine.

12. The method according to claim 4, wherein the AS₁ residue is the side chain of the amino acid valine and the AS₂ residue is the side chain of the amino acid valine.

13. The method according to claim 4, wherein the AS₁ residue is the side chain of the amino acid tryptophan and the AS₂ residue is the side chain of the amino acid valine.

14. The method according to claim 4, wherein the AS₁ residue is the side chain of the amino acid isoleucine and the AS₂ residue is the side chain of the amino acid valine.

15. The method according to claim 4, wherein the AS₁ residue is the side chain of the amino acid methionine and the AS₂ residue is the side chain of the amino acid valine.

16. The method according to claim 4, wherein the AS₁ residue is an H residue and the AS₂ residue is the side chain of the amino acid valine.

17. The method according to claim 1, wherein the compound is a compound selected from the following group of compounds:

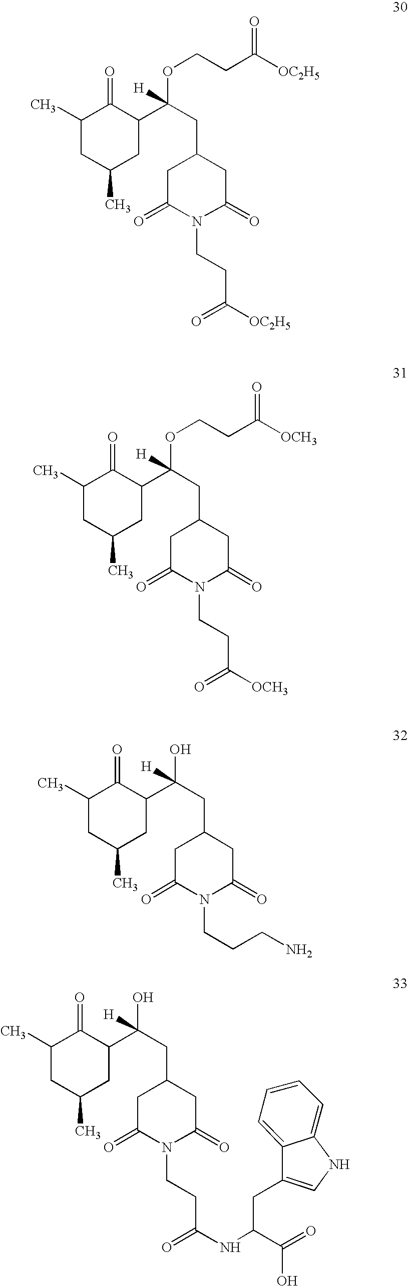

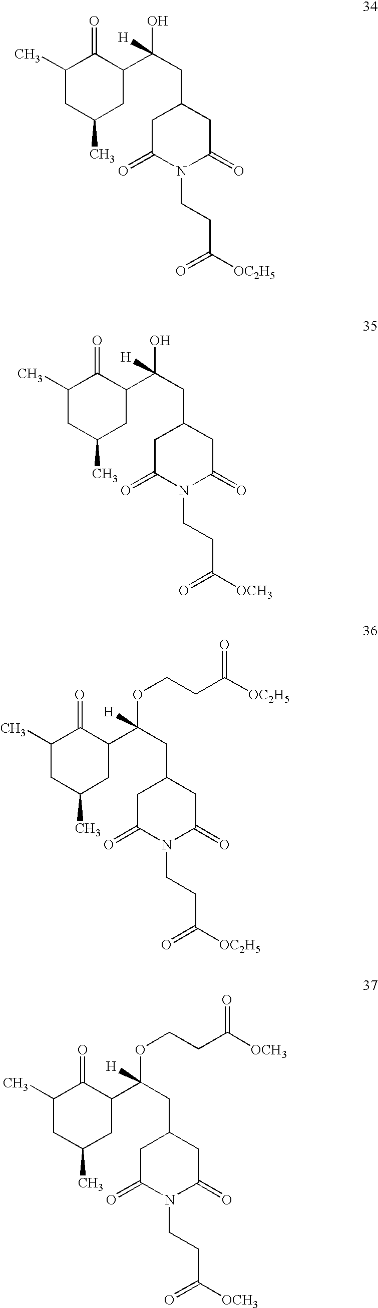

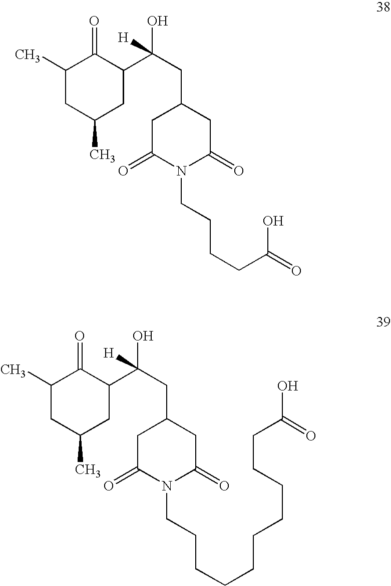

18. The method according to claim 1, wherein the cardiopathy is a damage caused to the myocardium by myocardial infarction, coronary insufficiency, myocardial infarction, angina pectoris, a coronary cardiopathy, an inflammatory disease of the cardiovascular system or of the myocardial muscle, in particular myocarditis, left-ventricular hypertrophy or dilated cardiomyopathy, in particular damage caused to the myocardium by myocardial infarction
and
the endothelial disease is damage to the endothelium, in particular damage to the endothelium caused by traumata, or an endothelial dysfunction
and
the traumata are traumata caused by surgical procedures, in particular traumata caused by transplantations, in particular traumata caused by angioplasty operations and
the lung disease is a chronic obstructive pulmonary disease, acute respiratory distress syndrome or mucoviscidosis
and
the vascular disease is angiospasm, in particular angiospasm in the case of Raynaud's disease, Prinzmetal's angina or in the case of ergotism, or coronary vasospasm, atherogenesis, atherosclerosis or a thrombotic disease
and
the form of the shock is an allergic, hypovolaemic or bacterial shock
and
the disease caused by ischaemia is myocardial infarction or ischaemic diseases or heart conditions or ischaemic conditions which trigger pain or are ischaemic conditions of peripheral organs or limbs.

19. The method of claim 1, wherein the transplants are organs.

20. The method of claim 1, wherein the diseases caused by ischaemic cell or tissue damage are necroses.
